(19) United States
(12) Patent Application Publication
(10) Pub. No.: US 2010/0198026 A1
(43) Pub. Date: Aug. 5, 2010

(54) PORTABLE MEASURING APPARATUS AND MEASURING METHOD FOR DETECTING SUNBURN

(75) Inventors: Nigel David Young, Redhill (GB); Peter Fairley, Newton Ferrers (GB); Hermann Christian Reccius, Cambridge (GB)

Correspondence Address: LIU & LIU, 444 S. FLOWER STREET, SUITE 1750, LOS ANGELES, CA, 90071, US

(73) Assignee: TPO Displays Corp., Chu-Nan (TW)

(21) Appl. No.: 12/645,225

(22) Filed: Dec. 22, 2009

Related U.S. Application Data
(60) Provisional application No. 61/206,715, filed on Feb. 2, 2009.

(51) Int. Cl. A61B 5/00

(52) U.S. Cl. 600/306

(57) ABSTRACT

A portable measuring apparatus for detecting sunburn is provided. The portable measuring apparatus includes a display module and a processing device coupled to the display module. The display module includes a light-emitting device emitting red, green and blue light to illuminate skin of a user, and a sensing device receiving and measuring reflected red, green and blue light from the skin of the user in response to the emitted red, green and blue light, respectively. The processing device obtains red, green and blue reflectance according to the reflected red, green and blue light, generates a sunburn index according to the red, green and blue reflectance and displays the sunburn index on the display module, wherein the sunburn index indicates onset of sunburn for the user.

### CROSS REFERENCE TO RELATED APPLICATIONS

This application claims the benefit of U.S. Provisional Application No. 61/206,715, filed on Feb. 2, 2009, the entirety of which is incorporated by reference herein.

### BACKGROUND OF THE INVENTION

1. Field of the Invention

The invention relates to a portable measuring apparatus, and more particularly to a portable measuring apparatus for detecting sunburn of a user.

2. Description of the Related Art

The exposure of human skin to physical agents present in solar ultraviolet (UV) radiation has been recognized as a health risk. The health risk to human skin has been exacerbated with urban heat island effects and global warming due to depletion of the ozone layer.

According to scientific research, too much exposure to UV radiation may damage a skin and sight of humans. For example, after being irradiated with strong UV rays, the epidermis produces chemical media and releases the chemical media to the derma, causing blood vessel dilatation and Erythema on the skin. As medical science research shows, the Erythema caused by UV rays is different from that caused by a burn. The Erythema caused by UV rays disappears very slowly, and may turn into black spots or induce skin cancer. Moreover, strong UV rays also damage eye tissues, cause conjunctivitis, keratitis, and damage crystalline lenses, which are reasons that induce cataracts.

Furthermore, although strong UV rays are usually present on sunny days, people may also suffer from too much exposure to UV rays on a cloudy day or even when indoors. In addition, some lamps also emit UV rays. Thus, exposure for humans is prevalent everywhere.

Currently, it is known that there is a direct relationship between a UV radiation dose in which a person has been exposed to and the probability for developing skin cancer. To prevent skin damage caused by sunburn, a UV radiation dose in which a person is exposed to must not be exceeded within a specific time interval. The UV radiation dose that is considered to be medically acceptable is called a minimal Erythema dose and is a radiation dose that leads to a visible red coloration after 24 hours. The value of the minimal Erythema dose depends on the skin type of the person.

Therefore, a portable measuring apparatus for direct detection of sunburn caused by UV rays is desired.

### BRIEF SUMMARY OF THE INVENTION

A portable measuring apparatus and a measuring method for detecting sunburn of a user are provided. An exemplary embodiment of a portable measuring apparatus for detecting sunburn of a user comprises a display module and a processing device coupled to the display module. The display module comprises a light-emitting device and a sensing device. The light-emitting device emits red, green and blue light to illuminate skin of the user. The sensing device receives and measures reflected red, green and blue light from the skin of the user in response to the emitted red, green and blue light, respectively. The processing device obtains red, green and blue reflectance according to the reflected red, green and blue light, respectively, generates a sunburn index according to the red, green and blue reflectance, and displays the sunburn index on the display module, wherein the sunburn index indicates onset of sunburn for the user.

Furthermore, an exemplary embodiment of a measuring method for detecting sunburn of a user by a portable apparatus with a display module and a processing device is provided. Red, green and blue light are emitted to illuminate skin of the user via the display module. Reflected red, green and blue light are received from the illuminated skin of the user in response to the emitted red, green and blue light via the display module, respectively. Red, green and blue reflectances are obtained according the reflected red, green and blue light. A sunburn index is obtained according to the red, green and blue reflectance and the sunburn index is displayed on the display module by the processing device, so as to indicate onset of sunburn for the user.

A detailed description is given in the following embodiments with reference to the accompanying drawings.

### BRIEF DESCRIPTION OF DRAWINGS

The invention can be more fully understood by reading the subsequent detailed description and examples with references made to the accompanying drawings, wherein:

FIG. 1 shows a diagram illustrating various reflectance distribution of human skin before and after UV irradiation;

FIG. 2 shows a portable measuring apparatus for detecting sunburn of a user according to an embodiment of the invention;

FIG. 3 shows an exemplary embodiment of a portable measuring apparatus for detecting sunburn of a user according to an embodiment of the invention; and

FIG. 4 shows a diagram illustrating the measured reflectance distribution of human skin according to the embodiment of FIG. 2.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

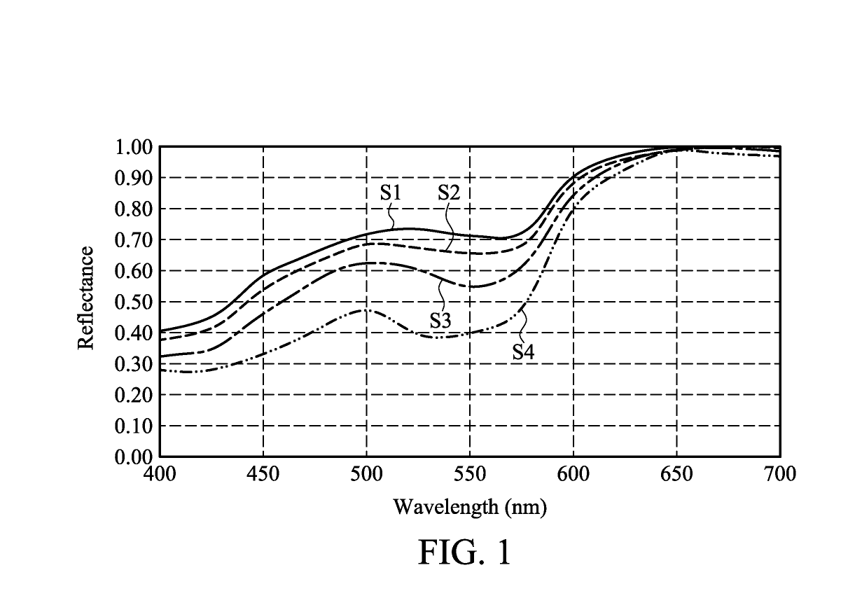

FIG. 1 shows a diagram illustrating various reflectance distribution of human skin before and after UV irradiation. In FIG. 1, curve S1 shows a reflectance spectrum of skin not exposed to UV irradiation, curve S2 shows a reflectance spectrum of skin mildly exposed to UV irradiation, curve S3 shows a reflectance spectrum of skin seriously exposed to UV irradiation, and curve S4 shows a reflectance spectrum of skin exposed to UV irradiation after 24 hours. The reflectance spectrum visible to the human eye begins at 420 nm, which is perceived by the eye as a blue shade, and reaches 700 nm, which corresponds to red shades, over the green shades. It is to be noted that the reflectance differences between the four curves are small for red wavelengths and the reflectance differences between the four curves are larger for green and blue wavelengths.

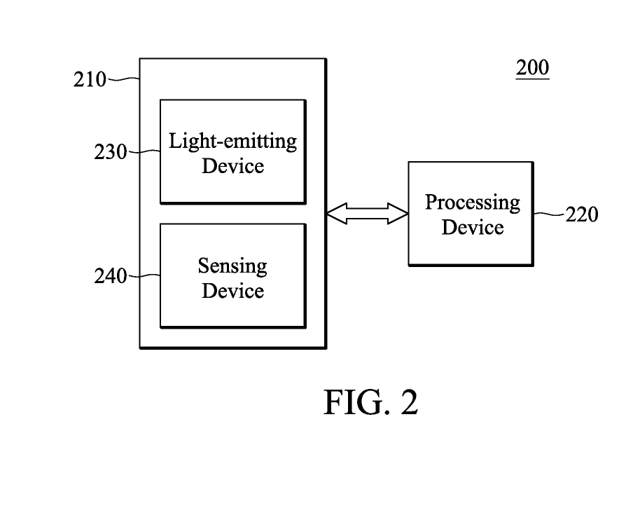

FIG. 2 shows a portable measuring apparatus 200 for detecting sunburn of a user according to an embodiment of the invention. The portable measuring apparatus 200 comprises a display module 210 and a processing device 220, wherein the display module 210 comprises a light-emitting device 230 and a sensing device 240. The light-emitting device 230 may be a display panel with red, green and blue pixels, such as a Light Emitting Diode (LED), a Liquid Crystal Display (LCD), an Organic Light Emitting Diode (OLED), and so on. The sensing device 240 comprises a plurality of photo-detectors which are disposed adjacent to the light-emitting device 230, disposed around a display area of the light-emitting device 230 or integrated into the pixels within the light-emitting device 230. The light-emitting device 230 is used as an illumination source which separately emits the red, green and blue light to the skin of the user, and then the sensing device 240 measures the reflected red, green and blue light from the skin of the user in response to the emitted red, green and blue light, respectively. For example, after the skin of the user is illuminated by the red light provided by the pixels corresponding to red color within the light-emitting device 230, the photo-detectors with optical filters corresponding to the red color are used to measure the reflected red light (i.e. the reflected light in response to the emitted red light). Next, the skin of the user is illuminated by the green and blue light in sequence, and the reflected green and blue light from the skin of the user are sequentially measured. Next, the processing device 220 may receive the measured results from the sensing device 240 to calculate the red, green and blue reflectance, and then to obtain a sunburn index indicating onset of sunburn for the user according to the red, green and blue reflectance. In one embodiment, the photo-detector may be a single photodiode without optical filters in order to reduce area and cost for the portable measuring apparatus 200. Thus, the reflected green and blue light from the skin of the user are sequentially measured by the single photodiode when the skin of the user is illuminated by the red, green and blue light in sequence. In another embodiment, the light-emitting device 230 may emit white light to the skin of the user, wherein the white light is composed of at least red, green and blue colors and may be provided by white LEDs. Due to the skin of the user is illuminated by the white light with all three colors simultaneously, the photo-detectors with optical filters corresponding to the red, green blue colors are used to measure the reflected red, green and blue light, respectively.

Moreover, the sunburn index may be an Erythema index (EI). The Erythema index is used to indicate a sunburn degree and is given by log₁₀(green reflectance/red reflectance). Furthermore, the sunburn index may be a Melanin index (MI), and an Infrared Ray (IR) source and IR photo-detectors are added to the display module 210 for emitting IR wavelengths and measuring IR reflectance, wherein the IR source and IR photo-detectors might be discrete components in the display module 210. Thus, the Melanin index is given by log₁₀(IR reflectance/red reflectance). After obtaining the sunburn index, the processing device 220 may display the sunburn index on the display area of the display module 210 for the user to view. In FIG. 2, the portable measuring apparatus 200 is a portable electronic apparatus with a display panel, such as a cellular phone, a Personal Digital Assistant (PDA), a Global Positioning System (GPS), a notebook and so on.

For the sensing device 240, the photo-detectors thereof may be discrete components in the display module 210 or thin film devices integrated into the Active Matrix (AM) array panel (e.g. the light-emitting device 230), such as vertical a-Si:H n-type-intrinsic-p-type (NIP) diodes, lateral low temperature poly-silicon (LTPS) diodes or thin-film transistors (TFTs) which may already be incorporated into the panel for ambient light control. In one embodiment, each photo-sensor may comprise three sub-components with separate optical filters corresponding to three primary colors, so as to enhance measurements. For example, the standard display red, green and blue color filters can be patterned over the three color pixels in an active matrix liquid crystal display (AMLCD). Sometimes, the photo-sensor may need to be light-shielded from direct illumination from the backlight so that interference from the backlight can be subtracted. Furthermore, if the photo-detectors are integrated into the pixels of the display module 210, the full display area of the display module 210 may be used to perform a detailed measurement of sunburn. When a photo-detector is integrated into a pixel corresponding to one of three primary colors, the photo-detector may comprise an optical filter with a specific wavelength bandwidth corresponding to the one of the three primary colors.

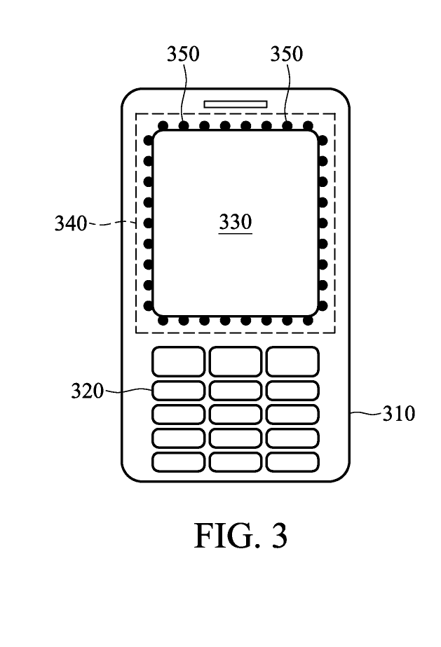

FIG. 3 shows an exemplary embodiment of a portable measuring apparatus for detecting sunburn of a user according to an embodiment of the invention. In FIG. 3, the portable measuring apparatus is a cellular phone 310. A keypad 320 and an AMLCD panel 330 are disposed on the surface of the cellular phone 310. In addition, a sensing device 340 disposed within the cellular phone 310 comprises a plurality of photo-detectors 350 around the AMLCD panel 330. In the embodiment, a processing device (not shown) is disposed within the cellular phone 310, such as below the AMLCD panel 330 or the keypad 320. By placing the AMLCD panel 330 of the cellular phone 310 to face on the skin of the user, a degree of sunburn according to the skin of the cheek of the user may be detected. For example, first, the processing device controls the AMLCD panel 330 to emit red light to illuminate the skin of the user, and then the processing device controls the photo-detectors 350 to receive and measure the reflected red light from the skin of the user in response to the emitted red light. Next, the processing device controls the AMLCD panel 330 to emit green light to illuminate the skin of the user, and controls the photo-detectors 350 to receive and measure the reflected green light from the skin of the user in response to the emitted green light. Final, the processing device controls the AMLCD panel 330 to emit blue light to illuminate the skin of the user, and controls the photo-detectors 350 to receive and measure the reflected blue light from the skin of the user in response to the emitted blue light. Each photo-sensor may perform a local measurement, and the measurement results can be used to represent skin variations for the user or to obtain an average result after eliminating extreme measurement result due to skin blemishes or measurement interferences caused by ambient optical paths.

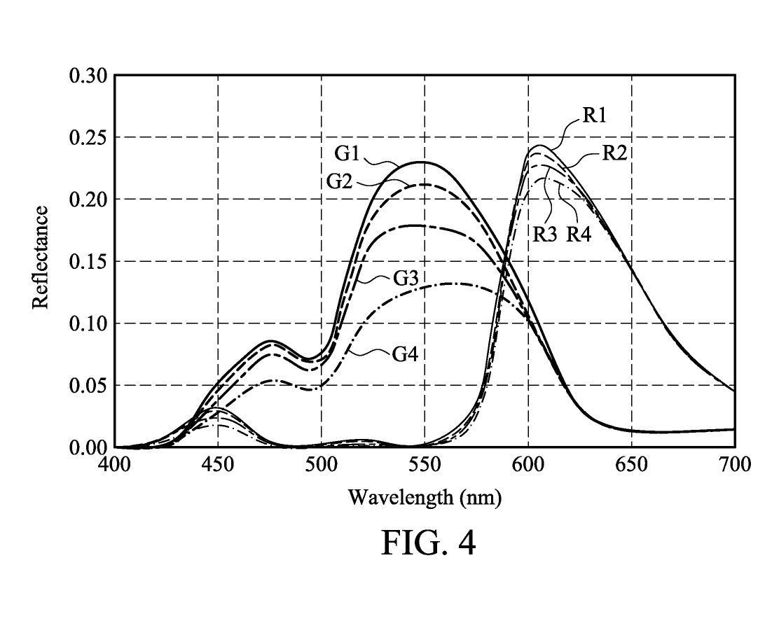

FIG. 4 shows a diagram illustrating the measured reflectance distribution of human skin according to the embodiment of FIG. 2, which indicates convolution of the back light spectra with the red and green color filters of the photo-detectors and the reflectance of the skin. In FIG. 4, curves R1, R2, R3 and R4 show the reflectance spectra with red color filter of skin not exposed to UV irradiation, skin mildly exposed to UV irradiation, skin seriously exposed to UV irradiation, and skin exposed to UV irradiation after 24 hours. Curves G1, G2, G3 and G4 show the reflectance spectra with green color filter of skin not exposed to UV irradiation, skin mildly exposed to UV irradiation, skin seriously exposed to UV irradiation, and skin exposed to UV irradiation after 24 hours. As described above, the reflectance differences during various exposure conditions are small at red wavelengths, thus the processing device may use the reflected red light to verify the measurement results. The verification is required because transmission of the entire optical path may vary due to external factors, such as the dirtied skin of the user or water or grease on the surface of the display panel. After the verification, the processing device 220 of FIG. 2 may obtain an Erythema index according to a ratio of the green reflectance to the red reflectance. Furthermore, the processing device 220 of FIG. 2 may use a ratio of the blue reflectance to the red reflectance to verify the Erythema index. For example, if the blue and green reflectance does not change by similar levels (as expected in FIG. 1), the value according to the Erythema index is determined to be an error value and the processing device 220 of FIG. 2 re-measures the reflectance.

While the invention has been described by way of example and in terms of preferred embodiment, it is to be understood that the invention is not limited thereto. Those who are skilled in this technology can still make various alterations and modifications without departing from the scope and spirit of this invention. Therefore, the scope of the present invention shall be defined and protected by the following claims and their equivalents.

What is claimed is:

1. A portable measuring apparatus for detecting sunburn of a user, comprising:
a display module, comprising:
a light-emitting device, emitting red, green and blue light to illuminate skin of the user; and
a sensing device, receiving and measuring reflected red, green and blue light from the skin of the user in response to the emitted red, green and blue light, respectively; and
a processing device coupled to the display module, obtaining red, green and blue reflectance according to the reflected red, green and blue light, respectively, generating a sunburn index according to the red, green and blue reflectance and displaying the sunburn index on the display module, wherein the sunburn index indicates onset of sunburn for the user.

2. The portable measuring apparatus as claimed in claim 1, wherein the light-emitting device is a display panel with red, green and blue pixels for separately emitting the red, green and blue light, and the sensing device comprises a plurality of photo-detectors for measuring the reflected red, green and blue light, so as to obtain the red, green and blue reflectance, respectively.

3. The portable measuring apparatus as claimed in claim 2, wherein the photo-detectors are disposed around a display area of the display panel or are disposed adjacent to the display panel.

4. The portable measuring apparatus as claimed in claim 2, wherein the photo-detectors are integrated into the red, green and blue pixels of the display panel.

5. The portable measuring apparatus as claimed in claim 2, wherein each of the photo-detectors comprises at least one optical filter with a specific wavelength

6. The portable measuring apparatus as claimed in claim 1, wherein the sunburn index is an Erythema index, and the processing device obtains the Erythema index according to a ratio of the green reflectance to the red reflectance.

7. The portable measuring apparatus as claimed in claim 6, wherein the processing device verifies the Erythema index according to a ratio of the blue reflectance to the red reflectance.

8. The portable measuring apparatus as claimed in claim 1, wherein the sensing device further receives a reflected infrared ray (IR) from the skin of the user to obtain an IR reflectance.

9. The portable measuring apparatus as claimed in claim 8, wherein the sunburn index is a Melanin index, and the processing device obtains the Melanin index according to a ratio of the IR reflectance to the red reflectance.

10. The portable measuring apparatus as claimed in claim 1, wherein the display module is an LED, an LCD or an OLED module.

11. A measuring method for detecting sunburn of a user by a portable apparatus with a display module and a processing device, comprising:
emitting red, green and blue light to illuminate skin of the user via the display module;
receiving reflected red, green and blue light from the illuminated skin of the user in response to the emitted red, green and blue light via the display module, respectively;
obtaining red, green and blue reflectance according the reflected red, green and blue light; and
obtaining a sunburn index according to the red, green and blue reflectance and displaying the sunburn index on the display module by the processing device, so as to indicate onset of sunburn for the user.

12. The measuring method as claimed in claim 11, wherein the red, green and blue light are separately emitted by a display panel of the display module via red, green and blue pixels therein, and the reflected red, green and blue light are received by a plurality of photo-detectors of the display module.

13. The measuring method as claimed in claim 12, wherein the photo-detectors are disposed around a display area of the display panel or are disposed adjacent to the display panel.

14. The measuring method as claimed in claim 12, wherein the photo-detectors are integrated into the red, green and blue pixels of the display panel.

15. The measuring method as claimed in claim 12, wherein each of the photo-detectors comprises at least one optical filter with a specific wavelength bandwidth corresponding to one of three primary colors.

16. The measuring method as claimed in claim 11, wherein the sunburn index is an Erythema index, and the Erythema index is obtained according to a ratio of the green reflectance to red the reflectance.

17. The measuring method as claimed in claim 16, wherein the Erythema index is verified by the processing device according to a ratio of the blue reflectance to the red reflectance.

18. The measuring method as claimed in claim 11, further comprising:
receiving a reflected infrared ray (IR) from the illuminated skin of the user to obtain an IR reflectance via the display module.

19. The measuring method as claimed in claim 18, wherein the sunburn index is a Melanin index, and the Melanin index is obtained according to a ratio of the IR reflectance to the red reflectance.

20. The measuring method as claimed in claim 11, wherein the display module is an LED, an LCD or an OLED module.
